(19) United States
(12) Patent Application Publication
(10) Pub. No.: US 2024/0108438 A1
(43) Pub. Date: Apr. 4, 2024

(54) FRICTION CONTROL FOR AN ORTHODONTIC APPLIANCE

(71) Applicant: SDC U.S. SmilePay SPV, Nashville, TN (US)

(72) Inventors: Tim Wucher, Windhoek (NA); Drew Marschner, Nashville, TN (US); Charlotte Xia, Nashville, TN (US); Justin Kreil, Nashville, TN (US); Ryan Ogletree, Nashville, TN (US)

(73) Assignee: SDC U.S. SmilePay SPV, Nashville, TN (US)

(21) Appl. No.: 17/957,985

(22) Filed: Sep. 30, 2022

(51) Int. Cl. A61C 7/08; A61C 7/00

(52) U.S. Cl. CPC A61C 7/08; A61C 7/002

(57) ABSTRACT

A device for repositioning a patient's teeth includes a shell having a shape corresponding to the patient's teeth and including an inner surface, where the inner surface is configured to engage with the patient's teeth. The device also includes at least one region of the inner surface having a higher frictional coefficient than a remaining portion of the inner surface, and where the at least one region is configured to apply a force to at least one tooth of the patient's teeth, wherein the at least one region comprises a local variation in geometry relative to a remainder of the inner surface.

### TECHNICAL FIELD

The present disclosure relates generally to the field of dental appliances. More specifically, the present disclosure relates to systems and methods for controlling friction between a patient's teeth and an orthodontic appliance.

### BACKGROUND

Dental aligners generated from physical or digital reproductions of a patient's teeth can be used by oral care professionals (e.g., dentists, orthodontists) to treat misalignment of the patient's teeth by repositioning the patient's teeth. Effectiveness of the treatment may often be affected by how well the dental aligner controls movement of each of the patient's teeth such that the dental aligner moves the patient's teeth as specified by a treatment plan. Accordingly, it would be advantageous to provide a method and device for enhancing control between the dental aligner and a surface of the patient's teeth to move the patient's teeth with greater efficiency and accuracy, and to ensure the patient's teeth move according to their treatment plan.

### SUMMARY

One embodiment of the present disclosure relates to a device for repositioning a patient's teeth. The device includes a shell having a shape corresponding to the patient's teeth and including an inner surface, where the inner surface is configured to engage with the patient's teeth. The device also includes at least one region disposed on the inner surface, where the at least one region has a higher frictional coefficient than a remaining portion of the inner surface, and where the at least one region is configured to apply a force to at least one tooth of the patient's teeth, wherein the at least one region comprises a local variation in geometry relative to a remainder of the inner surface.

Another aspect of the present disclosure relates to an orthodontic system for repositioning a patient's teeth. The system includes an aligner having an inner surface, where the inner surface is configured to engage with the patient's teeth and has a shape corresponding to the patient's teeth. The aligner also includes at least one region of the inner surface having a plurality of protrusions extending outwardly from the inner surface, and where each protrusion of the plurality of protrusions is configured to at least one tooth of the patient's teeth, wherein the plurality of protrusions include a first protrusion having a first length and a second protrusion having a second length greater than the first length.

Yet another aspect of the present disclosure relates to a method of repositioning a patient's teeth. The method includes generating, by one or more processors of a treatment planning computer system, a representation of the patient's teeth. The method further includes identifying, by the one or more processors based on the representation, a location on at least one tooth of the patient's teeth for applying a force to cause a positional adjustment of the at least one tooth. The method further includes determining, by the one or more processors based on the location, a corresponding region on an inner surface of an aligner to be manufactured to apply the force to the location. The method further includes selecting, by the one or more processors, a surface modification for the corresponding region, where the surface modification comprises a plurality of protrusions. The method further includes generating, by the one or more processors, a treatment plan for the patient to cause the position adjustment of the at least one tooth, where the treatment plan includes applying the surface modification to the corresponding region of the aligner.

Various other embodiments and aspects of the disclosure will become apparent based on the drawings and detailed description of the following disclosure.

### BRIEF DESCRIPTION OF THE FIGURES

FIG. 1 is a system for orthodontic treatment including a treatment planning computer system, according to an exemplary embodiment.

FIG. 2A is a perspective view of an orthodontic appliance and a patient's oral cavity, according to an exemplary embodiment.

FIG. 2B is a top view of an orthodontic appliance, according to an exemplary embodiment.

FIG. 3 is a schematic representation of a surface texture profile for an interior surface of the orthodontic appliance of FIG. 2A, according to an exemplary embodiment.

FIG. 4 is a schematic representation of another surface texture profile for an interior surface of the orthodontic appliance of FIG. 2A, according to an exemplary embodiment.

FIG. 5 is a top view of a portion of the orthodontic appliance of FIG. 2A, showing a surface texture of an inner surface of the aligner, according to an exemplary embodiment.

FIG. 6 is a perspective view of a surface having a second surface texture within the orthodontic appliance of FIG. 2A, according to an exemplary embodiment.

FIG. 7 is side view of a surface having a third surface texture within the orthodontic appliance of FIG. 2A, according to an exemplary embodiment.

FIG. 8 is a perspective view of a surface having a fourth surface texture within the orthodontic appliance of FIG. 2A, according to an exemplary embodiment.

FIG. 9 is a perspective view of a surface having a fifth surface texture within the orthodontic appliance of FIG. 2A, according to an exemplary embodiment.

FIG. 10 is a perspective view of a surface having a sixth surface texture within the orthodontic appliance of FIG. 2A, according to an exemplary embodiment.

FIG. 11 is a perspective view of a surface having a seventh surface texture within the orthodontic appliance of FIG. 2A, according to an exemplary embodiment.

FIG. 12 is a block diagram of a method of repositioning the patient's teeth by implementing friction control in the orthodontic appliance of FIG. 2A, according to an exemplary embodiment.

### DETAILED DESCRIPTION

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

As described above, orthodontic appliances or devices (e.g., aligners) may be used to treat misalignment of the teeth by applying one or more forces to individual teeth, which causes the teeth to adjust their relative position within an oral cavity when one or more forces are applied over time. To effectively apply the one or more forces, it is imperative that the aligner has a sufficiently high friction of resistance such that the aligner effectively grips each tooth to effectuate accurate and sufficient movement of the tooth. Although various methods exist for gripping teeth, these methods often involve installation of various attachments, brackets, buttons, or other fixtures to one or more of the patient's teeth, which allow the aligner to be anchored thereon. Such methods tend to be highly invasive, which may cause discomfort or enamel damage to patients receiving orthodontic treatment and which may be aesthetically unappealing. Other methods have involved permanent or semi-permanent bonding of the aligner to patients' teeth, or manipulating a stiffness of the aligner to minimize elasticity (e.g., make the aligner more rigid and less prone to deformation). However, these strategies may require extensive preparation and can result in aligner arrangements within the patient's oral cavity that are uncomfortable or that have minimal effectiveness.

Accordingly, the present disclosure relates to systems and methods for controlling tooth positioning in a manner that is minimally invasive to a patient and provides customizable aligner configurations for facilitating force application to reposition teeth. Generally, the figures illustrate systems and methods for controlling friction between a surface of a patient's teeth and an inner surface of an orthodontic appliance to facilitate and enhance accurate control of tooth movement during orthodontic treatment. In various embodiments, an orthodontic appliance may include one or more regions on an interior surface of the appliance that are structured to increase a coefficient of friction between the orthodontic appliance and a surface of an adjacent tooth. As such, the aligner is better able to grip the surfaces of teeth, thereby reducing slippage between the aligner and the teeth and resulting in more accurate and predictable treatment outcomes. In some embodiments, the one or more regions may include a plurality of hierarchical protrusions configured to engage with contours and/or asperities within the tooth surface. In some embodiments, the one or more regions may include one or more composites, particles, fibers, cilia, or other features configured to engage with the tooth surface and apply a threshold amount of friction to the tooth surface. In some embodiments, the one or more regions may include topological features including, but not limited to, grooves and/or ridges, that are configured to alter frictional and/or normal forces applied to the tooth surface. In some instances, the orthodontic appliance may be used in combination with one or more viscous mediums, such as a gel or paste, which may increase a coefficient of friction between the inner surface of the dental appliance and the tooth surface (i.e., by increasing a coefficient of friction between the one or more regions and the tooth surface).

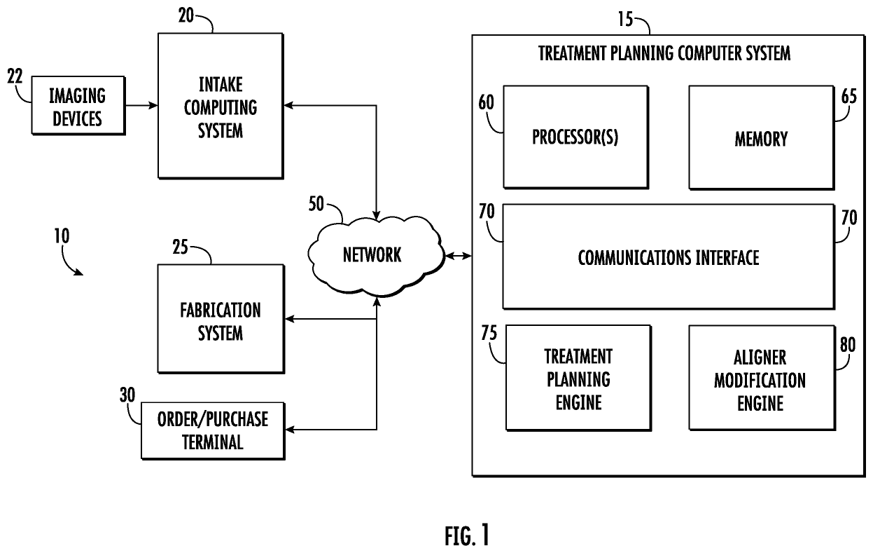

Referring to FIG. 1, a system 10 for orthodontic treatment is shown, according to an illustrative embodiment. As shown in FIG. 1, the system 10 includes a treatment planning computer system 15 communicably coupled to an intake computing system 20, and a fabrication system 25. In some embodiments, the treatment planning computer system 15 may be or may include one or more servers which are communicably coupled to a plurality of computing devices. In some embodiments, the treatment planning computer system 15 may include a plurality of servers, which may be located at a common location (e.g., a server bank) or may be distributed across a plurality of locations. The treatment planning computer system 15 may be communicably coupled to the intake computing system 20, fabrication system 25, and order/purchase terminal 30, via a communications link or network 50 (which may be or include various network connections configured to communicate, transmit, receive, or otherwise exchange data between addresses corresponding to the computing systems 15, 20, 25, 30). The network 50 may be a Local Area Network (LAN), a Wide Area Network (WAN), a Wireless Local Area Network (WLAN), an Internet Area Network (IAN) or cloud-based network, etc. The network 50 may facilitate communication between the respective components of the system 10, as described in greater detail below.

The computing systems 15, 20, 25, 30 include one or more processing circuits, which may include processor(s) 60 and memory 65. The processor(s) 60 may be a general purpose or specific purpose processor, an application specific integrated circuit (ASIC), one or more field programmable gate arrays (FPGAs), a group of processing components, or other suitable processing components. The processor(s) 60 may be configured to execute computer code or instructions stored in memory 65 or received from other computer readable media (e.g., CDROM, network storage, a remote server, etc.) to perform one or more of the processes described herein. The memory 65 may include one or more data storage devices (e.g., memory units, memory devices, computer-readable storage media, etc.) configured to store data, computer code, executable instructions, or other forms of computer-readable information. The memory 65 may include random access memory (RAM), read-only memory (ROM), hard drive storage, temporary storage, non-volatile memory, flash memory, optical memory, or any other suitable memory for storing software objects and/or computer instructions. The memory 65 may include database components, object code components, script components, or any other type of information structure for supporting the various activities and information structures described in the present disclosure. The memory 65 may be communicably connected to the processor 60 via the processing circuit, and may include computer code for executing (e.g., by processor(s) 60) one or more of the processes described herein.

The treatment planning computer system 15 is shown to include a communications interface 70. The communications interface 70 can be or can include components configured to transmit and/or receive data from one or more remote sources (such as the computing devices, components, systems, and/or terminals described herein). In some embodiments, each of the servers, systems, terminals, and/or computing devices may include a respective communications interface 70 which permit exchange of data between the respective components of the system 10. As such, each of the respective communications interfaces 70 may permit or otherwise enable data to be exchanged between the respective computing systems 15, 20, 25, 30. In some implementations, communications device(s) may access the network 50 to exchange data with various other communications device(s) via cellular access, a modem, broadband, Wi-Fi, satellite access, etc. via the communications interfaces 70.

The treatment planning computer system 15 is shown to include a plurality of engines, including a treatment planning engine 75 and an aligner modification engine 80. The treatment planning engine 75 and the aligner modification engine 80 may be any device(s), component(s), circuit(s), or other combination of hardware components designed or implemented to receive inputs for and/or automatically generate a treatment plan implementing dental appliances (e.g., dental aligners configured to reposition one or more teeth of the patient) including modified surfaces configured to reposition one or more teeth of the patient more efficiently and with more control over traditional aligner surfaces. In some embodiments, the treatment planning engine 75 and the aligner modification engine 80 may be instructions stored in memory 65 which are executable by the processor(s) 60. In some embodiments, the treatment planning engine 75 and the aligner modification engine 80 may be stored at the treatment planning computer system 15 and accessible via a respective treatment planning terminal.

The intake computing system 20 may be configured to generate a 3D model (e.g., a 3D digital model) of a dentition. In some embodiments, the intake computing system 20 may be communicably coupled to or otherwise include one or more imaging devices 22 configured to generate, capture, or otherwise produce a 3D model 300 of an object, such as a dentition or dental arch. In some embodiments, the imaging devices 22 may include intraoral scanners configured to generate a 3D model of a dentition of a patient as the intraoral scanner passes over the dentition of the patient. For example, the intraoral scanner may be used during an intraoral scanning appointment, such as the intraoral scanning appointments described in U.S. Provisional Patent Application No. 62/660,141, titled “Arrangements for Intraoral Scanning,” filed Apr. 19, 2018, and U.S. patent application Ser. No. 16/130,762, titled “Arrangements for Intraoral Scanning,” filed Sep. 13, 2018, the contents of each of which are incorporated herein by reference in their entirety. In some embodiments, the imaging devices 22 may include 3D scanners configured to scan a dental impression. The dental impression may be captured or administered by a patient using a dental impression kit similar to the dental impression kits described in U.S. Provisional Patent Application No. 62/522,847, titled “Dental Impression Kit and Methods Therefor,” filed Jun. 21, 2017, and U.S. patent application Ser. No. 16/047,694, titled “Dental Impression Kit and Methods Therefor,” filed Jul. 27, 2018, the contents of each of which are incorporated herein by reference in their entirety. In these and other embodiments, the imaging devices 22 may generally be configured to generate a 3D model of a dentition of a patient. As an example, the 3D model may be a point cloud representation of the dentition, a voxel representation, a spline representation, a mesh representation, or any other parametric model representation. In some embodiments, the imaging devices 22 may be configured to capture a two dimensional (2D) image of a dentition of the patient that is then converted into a 3D model. For example, the imaging devices 22 can be a user device, such as a smartphone, tablet, or camera. The imaging devices 22 may be configured to generate a 3D model of the upper (i.e., maxillary) dentition and/or the lower (i.e., mandibular) dentition of the patient. One or more 2D images can be converted into a digital model using any of the systems and processes disclosed in U.S. patent application Ser. No. 16/696,468, titled “Systems and Methods for Constructing a Three-Dimensional Model from Two-Dimensional Images,” filed Nov. 26, 2019, and U.S. patent application Ser. No. 17/247,055, titled “Systems and Methods for Constructing a Three-Dimensional Model from Two-Dimensional Images,” filed Nov. 25, 2020, the contents of each of which are incorporated herein by reference in their entirety. The 3D model may include a digital representation of the patient's teeth and/or gingiva. The imaging devices 22 may be configured to generate 3D models of the patient's dentition prior to treatment (i.e., with their teeth in an initial position). In some embodiments, the imaging devices 22 may be configured to generate the 3D models of the patient's dentition in real-time (e.g., as the dentition/impression is imaged). In some embodiments, the imaging devices 22 may be configured to export, transmit, send, or otherwise provide data obtained during the imaging to an external source which generates the 3D model, and transmits the 3D model to the intake computing system 20.

The intake computing system 20 may be configured to transmit, send, or otherwise provide the 3D model to the treatment planning computer system 15. In some embodiments, the intake computing system 20 may be configured to provide the 3D model of the patient's dentition to the treatment planning computer system 15 by uploading the 3D model to a patient file for the patient. The intake computing system 20 may be configured to provide the 3D model of the patient's upper and/or lower dentition at their initial (i.e., pre-treatment) position. The 3D model of the patient's upper and/or lower dentition may together form initial imaging data which represents an initial position of the patient's teeth prior to treatment.

The treatment planning engine 75 is configured to generate a treatment plan based on or using the 3D model created from the imaging data. The treatment planning engine 75 is configured to modify, correct, adjust, or otherwise process the initial imaging data or 3D model received from the intake computing system 20 prior to generating a treatment plan. The treatment planning engine 75 is configured to define a gingival line of the 3D model such that the teeth can be separated from the gingiva shown in the 3D model. The treatment planning engine 75 is configured to segment individual teeth from the tooth model to separate the teeth from one another. The treatment planning engine 75 is configured to generate stages of treatment (e.g., a treatment plan) for one or more of the patient's teeth to move relative to one another from an initial position to a final position. As part of staging, the treatment planning engine 75 is configured to generate a plurality of staged 3D models corresponding to the treatment plan for the patient. Each 3D model may be representative of a particular stage of the treatment plan (e.g., a first 3D model corresponding to an initial stage of the treatment plan, one or more intermediate 3D models corresponding to intermediate stages of the treatment plan, and a final 3D model corresponding to a final stage of the treatment plan).

The aligner modification engine 80 is configured to determine and select modifications to be made to the dental aligners to cause the dental aligners to grip the teeth, and therefore exert a sufficient force on the teeth, intended to be repositioned by the treatment plan. For example, the aligner modification engine 80 can be configured to select from among a plurality of possible surface treatments and modifications to modify (e.g., increase or decrease) the coefficient of friction between the orthodontic appliance and a surface of an adjacent tooth. In some cases, the aligner modification engine 80 may select a possible surface treatment to increase the coefficient of friction. In some examples the aligner modification engine 80 may select a surface treatment to decrease the coefficient of friction. For example, in various embodiments, the aligner modification engine 80 may select a first surface treatment for a first portion of an aligner to increase friction to increase resistance of tooth movement relative to the first portion, and the aligner modification engine 80 may select a second surface treatment for a second portion of the aligner to decrease friction to thus decrease resistance of tooth movement relative to the second portion. In various embodiments, reducing the coefficient of friction in some regions facilitates movement of teeth in those regions, which allows for repositioning of the teeth (e.g., in adherence with a predetermined treatment plan). In some embodiments, the surface treatment modifies one or more regions of the aligner such that the coefficient of friction falls between a first and second threshold, such that the coefficient of friction falls below a first threshold but remains above a second threshold value, where the first threshold value defines a maximum acceptable coefficient of friction and the second threshold value defines a minimum acceptable coefficient of friction. The surface treatments and modifications can include a local variation in geometry (i.e., relative to a remainder of the inner surface 124). The local variation in geometry may include a plurality of geometric surface features, such as hierarchical protrusions and/or cavities, which are configured to engage with contours and/or asperities within the tooth surface. The surface treatments may additionally or alternatively include one or more composites, particles, fibers, cilia, or other features configured to engage with the tooth surface and apply a threshold amount of friction to the tooth surface as required by the treatment plan. The surface treatments (i.e., including the local variation in geometry) may additionally or alternatively include other topological features such as grooves and/or ridges that are configured to alter frictional and/or normal forces applied to the tooth surface, and so on as discussed herein.

The aligner modification engine 80 is configured to analyze the digital or physical representation of the teeth 102 to determine at least one tooth among the patient's teeth 102 that is to be repositioned and determine a corresponding location (e.g., region 20, 108, 110 and/or 112) on the teeth 102 for applying a force to effectuate movement of the at least one tooth.

The aligner modification engine 80 is configured to determine corresponding locations (e.g., regions 126, 128, 130, and/or 132) within an aligner (e.g., the aligner 120) to apply one or more forces to the teeth 102 or resist movement of the teeth 102 to facilitate positional adjustment thereof. The aligner modification engine 80 is configured to select a desired surface texture or surface type for each of the one or more locations within the aligner where a force is to be applied or movement of the teeth 102 is to be increased or decreased within the aligner. The aligner modification engine 80 can select the desired surface texture or surface type by determining a particular configuration of protrusions 140, such as by selecting the height 143, the distance 147, the angles 150, 155, an orientation or configuration of the portion 157, a number of protrusions 140, etc. In some embodiments, selecting the desired surface texture or surface type may additionally or alternatively include selecting a surface treatment for the one or more locations (e.g., region 126, 128, 130 and/or 132), which may include etching, micromolding, 3D printing, composite or particulate deposition, etc.

The treatment planning computer system 15 may be configured to transmit, send, or otherwise provide the staged 3D models including modifications determined by the aligner modification engine 80 to the fabrication system 25. In some embodiments, the treatment planning computer system 15 may be configured to provide the staged 3D models and the modifications to the fabrication system 25 by uploading the staged 3D models and modifications to a patient file which is accessible via the fabrication system 25. In some embodiments, the treatment planning computer system 15 may be configured to provide the staged 3D models and modifications to the fabrication system 25 by sending the staged 3D models and modifications to an address (e.g., an email address, IP address, etc.) for the fabrication system 25.

The fabrication system 25 can include a fabrication computing device and fabrication equipment configured to produce, manufacture, or otherwise fabricate dental aligners. The fabrication system 25 may be configured to receive a plurality of staged 3D models and modifications corresponding to the treatment plan for the patient. The fabrication system may be configured to send the staged 3D models and modifications to fabrication equipment for generating, constructing, building, or otherwise producing dental aligners 120 that can be worn by the patient to reposition one or more teeth of the patient. In some embodiments, the fabrication equipment may include a 3D printing system. The 3D printing system may be used to 3D print physical models corresponding the 3D models of the treatment plan. As such, the 3D printing system may be configured to fabricate physical models which represent each stage of the treatment plan. The fabrication equipment 218 may also include a thermoforming system. The thermoforming system may be configured to thermoform a polymeric material to the physical models, and cut, trim, or otherwise remove excess polymeric material from the physical models to fabricate a dental aligner. In some embodiments, the 3D printing system may be configured to directly fabricate dental aligners 120 (e.g., by 3D printing the dental aligners 120 directly based on the 3D models of the treatment plan). Additional details corresponding to fabricating dental aligners 120 are described in U.S. Provisional Patent Application No. 62/522,847, titled “Dental Impression Kit and Methods Therefor,” filed Jun. 21, 2017, and U.S. patent application Ser. No. 16/047,694, titled “Dental Impression Kit and Methods Therefor,” filed Jul. 27, 2018, and U.S. patent application Ser. No. 16/188,570, titled “Systems and Methods for Thermoforming Dental Aligners,” filed Nov. 13, 2018, the contents of each of which are incorporated herein by reference in their entirety.

The fabrication equipment may be configured to generate or otherwise fabricate dental aligners 120 for each stage of the treatment plan. In some instances, each stage may include a plurality of dental aligners 120 (e.g., a plurality of dental aligners 120 for the first stage of the treatment plan, a plurality of dental aligners 120 for the intermediate stage(s) of the treatment plan, a plurality of dental aligners 120 for the final stage of the treatment plan, etc.). Each of the dental aligners 120 may be worn by the patient in a particular sequence for a predetermined duration (e.g., two weeks for a first dental aligner 120 of the first stage, one week for a second dental aligner 120 of the first stage, etc.).

The order/purchase terminal 30 may include any device(s), component(s), circuit(s), or other combination of hardware components designed or implemented to complete and/or guide a user in placing an order. An order may be a transaction that exchanges money from a patient for a product (e.g., an impression kit, dental aligners, etc.). The order/purchase terminal 30 may communicate with the fabrication system 25 and third party device (e.g., a patient device or other user device) to guide a patient or other user through a payment/order completion system without requiring the patient to order through a traditional dentist office. In some embodiments, the order/purchase terminal 30 may communicate prompts to the user device to guide the user through the payment/order completion system. The prompts may include asking the patient for patient information (e.g., name, physical address, email address, phone number, credit card information) and product information (e.g., quantity of product, product name). In response to receiving information from the patient, the order/purchase terminal 30 initiates a product order. The initiated product order is transmitted to the fabrication system 25 to initiate the fabrication of one or more products (e.g., dental aligners). The initiated product order may also be transmitted to the intake computing system 20 to store/record the transaction and/or to initiate a product order from the computing system (e.g., a dental impression kit, dental aligners, etc.), and the like.

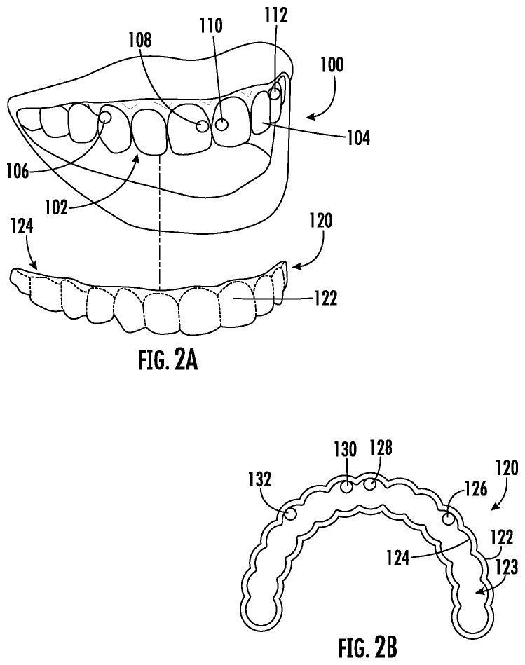

Referring to FIG. 2A, a dental appliance (“aligner”) 120 configured for adjusting a position of one or more teeth 102 within a patient's oral cavity is shown. In various embodiments, the aligner 120 includes a polymeric shell having an outer surface 122 and an inner surface 124, which interfaces with the patient's teeth 102, where the aligner 120 has a shape that substantially corresponds to a shape of the teeth 102. The aligner 120 is structured to apply one or more forces to a surface 104 of the teeth 102 in accordance with a treatment plan approved by an orthodontic professional (e.g., a licensed dentist or orthodontist). In various embodiments, the inner surface 124 of the aligner 120 may be made from the same material or have substantially equivalent mechanical and physical properties as the outer surface 122. In some embodiments, the inner surface 124 may be made from a different material or have substantially different mechanical and physical properties as the outer surface 122. For example, the inner surface 124 may be made from a softer (e.g., having a lower elastic modulus) material as compared to the outer surface 122.

In various embodiments, a 3D model of the patient's teeth 102 may be obtained. For example, the 3D model can be obtained by conducting an intraoral scan of the patient's teeth, by processing images (e.g., 2D images or scan images) of a dental impression taken of the patient's teeth, or by converting 2D images of the patient's teeth into a 3D model. A treatment plan is generated based on the 3D model by the treatment planning computer system 15, and the treatment plan can specify specific regions of the aligner 120 that will interface with specific regions of the teeth 102 and in turn apply force to the teeth 102. As shown in FIG. 2A, regions, 106, 108, 110, and 112, which are disposed on the surface 104 of the teeth 102 may be identified as regions corresponding to where forces may be applied by the aligner 120. Although FIG. 2A shows four regions, in various implementations, any number of regions may be determined (e.g., 1, 2, 3, 5, 15, 115, etc.).

As shown in FIG. 2B, the aligner 120 is structured such that the inner surface 124 is curved to define an inner contact area 123, which is configured to at least partially surround the patient's teeth 102 during orthodontic treatment to facilitate the application of force thereto. Accordingly, to reduce slippage between the aligner 120 and each of the regions 106, 108, 110, and 112, the aligner 120 (e.g., the area 123) may be structured to include regions along the inner surface 124 of the aligner corresponding to the regions on the tooth surface 104 having a relatively higher coefficient of friction. In some embodiments, only these specific regions have a higher coefficient of friction and the remaining portions of the aligner 120 are not modified. In some embodiments, the entire inner surface 124 is modified to have a higher coefficient of friction. In some embodiments, the specific regions being modified to have a higher coefficient of friction target specific teeth. As shown in FIG. 2B, the inner surface 124 may include regions 126, 128, 130, and 132, which respectively correspond to the regions 20, 108, 110, and 112 on the tooth surface 104. The regions 126, 128, 130, and 132 are configured to apply a threshold amount of force to the teeth 102 by having a higher coefficient of friction as compared to a coefficient of friction in other areas within the inner surface 124 that are not within the regions 126, 128, 130, 132.

Although FIG. 2B shows the four regions 126, 128, 130, and 132, in various embodiments, the aligner 120 may be structured to include any number of regions (e.g., 1, 2, 3, 5, 115, etc.). In some embodiments, all of (or nearly all of) the inner surface 124 may be configured to apply the threshold amount of force to the teeth 102 by having a higher coefficient of friction, thereby enabling the aligner 120 to sufficiently grip the teeth 102 for applying the requisite force, and resist movement with respect to the teeth 102 (i.e., reduce slippage of the aligner on the teeth). In various embodiments, each of the regions 126, 128, 130, and 132 may be separate, discrete regions disposed on the inner surface 124. In some embodiments, two or more of the regions 126, 128, 130, and 132 may be adjacent or conjoined.)

In some embodiments, each of the regions 126, 128, 130, and 132, which are disposed along the inner surface 124, may include one or more surface features or textures configured to apply a threshold amount of force to the teeth 102 by having a higher coefficient of friction to facilitate adequate gripping of the teeth 102 and to prevent or resist movement between the aligner 120 and the teeth 102 (i.e. reduce slippage of the aligner on the teeth). FIG. 3 shows a schematic representation of a portion of the inner surface 124 (e.g., which may be within one of the regions 126, 128, 130, and 132). As shown, the inner surface 124 may include one or more protrusions 140 (e.g., stalks, fibers, cilia, etc.), which may extend away from the inner surface 124. Each of the protrusions 140 may be structured to engage with one or more surface features (e.g., asperities) within the surface 104 of the teeth 102 to increase a coefficient of friction between the inner surface 124 and the teeth 102 (e.g., by increasing a contact surface area between the inner surface 124 and the teeth 102) and reduce slippage therebetween. In various embodiments, a height 143 of the protrusions 140 (or a depth of ridges or grooves formed between adjacent protrusions 140) may correspond to a depth of a ridge or asperity of the teeth 102 (e.g., on an individual tooth). In some embodiments, the height 143 may be based on a desired direction of force application to the teeth 102. In various embodiments, the height 143 may have a numerical value on the order of microns. For example, in various embodiments, the height 143 may have a magnitude up to approximately 500 microns. In some embodiments, the height 143 may have a numerical value on the order of nanometers. In some embodiments, the height 143 may have a numerical value on the order of millimeters.

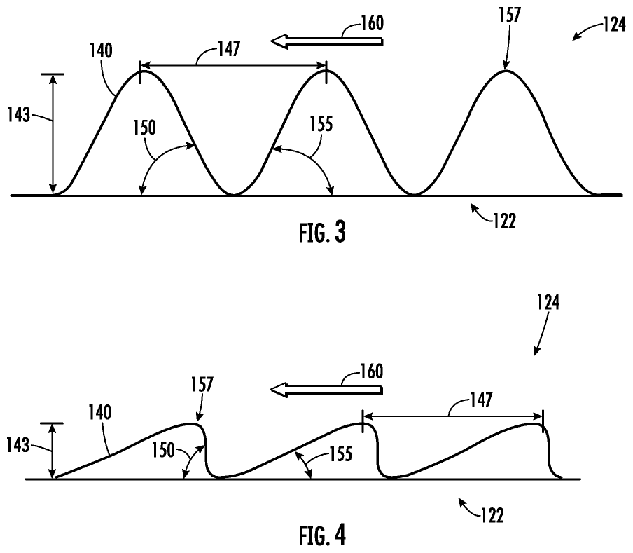

As shown in FIG. 3, each of the protrusions 140 may be oriented at an angle relative to the inner surface 124. For example, each protrusion 140 may be oriented at a first angle 150 on a first side of the protrusion 140 and at a second angle 155 on a second side of the protrusion. In various embodiments, the angles 150, 155 may be customizable (e.g., during fabrication of the aligner 120 and as determined by the treatment planning computer system 15) to adjust an orientation of the protrusions 140. For example, as shown in FIG. 4, the protrusions 140 may be arranged such that the first angle 150 is greater than the second angle 155 such that the protrusions tilt or lean relative to the inner surface 124. In some embodiments, such as shown in FIG. 3, the first angle 150 may be substantially equal to the second angle 155 such that each of the protrusions 140 is oriented substantially perpendicular to the inner surface 124. In various embodiments, the protrusions 140 may be structured such that at least one of the first angle 150 or the second angle 155 is based on a desired force to be applied to a tooth or a resistive force necessary to overcome slippage of the inner surface 124 (and thus the aligner 120) in a direction 160 relative to the movement of the tooth.

As shown in FIGS. 3 and 4, the protrusions 140 may be arranged such that individual protrusions 140 are disposed in an adjacent fashion along the inner surface 124 in rows, clusters, columns, arrays, etc. Each of the protrusions 140 may be spaced from adjacent protrusions 140 by a distance 147. In some embodiments, the distance 147 may be defined as a distance between an uppermost portion (e.g., tip) 157 of adjacent protrusions 140. In some embodiments, the distance 147 may be defined as a distance between a lowermost portion (e.g., base) of adjacent protrusions 140. In various embodiments, the protrusions 140 may be structured such that the distance 147 may correspond to or be based on a surface structure or topology of teeth 102. For example, the distance 147 may correspond to a ridge spacing on the surface 104 or a width of an individual tooth.

In some embodiments, at least one of the distance 147, the height 143, the angles 150, 155, or a number of the protrusions 140 may be based on location of the regions 126, 128, 130, 132. In some embodiments, at least one of the distance 147, the height 143, the angles 150, 155, or a number of the protrusions 140 may be based on a desired amount of repositioning of the teeth 102. In some embodiments, at least one of the distance 147, the height 143, the angles 150, 155, or a number of the protrusions 140 may be based on one or more features on the surface 104 as determined by the treatment planning computer system 15. For example, the treatment planning computer system 15 may determine a desired configuration for the aligner 120, including the number of regions (e.g., each similar or equivalent to the regions 126, 128, 130, 132), and a number and arrangement of the protrusions 140 along the inner surface 124 based on the movements required to ultimately move the teeth 102 from an initial position to a final position.

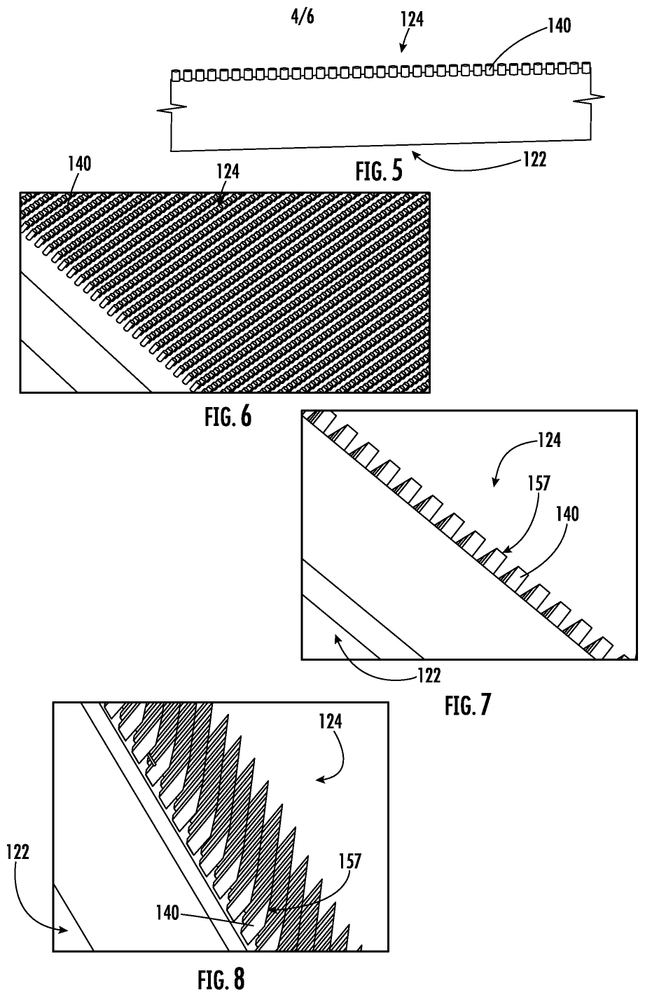

In various embodiments, the protrusions 140 may be disposed in a grid-like arrangement, such as shown in FIGS. 5 and 6. Accordingly, the distance 147 between adjacent protrusions 140 may be constant (e.g., along the inner surface 124 or within each of the regions 126, 128, 130, 132). In some embodiments, the height 143 of each protrusion 140 may be the same. In some embodiments, the inner surface 124 (and thus the regions 126, 128, 130, 132) may be structured such that the protrusions 140 have varied heights 143 (e.g., have a hierarchical configuration). In some embodiments, the protrusions 140 may be arranged in a hierarchical configuration such that at least one of the heights 143 of each individual protrusion 140 or the distance 147 between adjacent protrusions 140 may be based on a feature of the surface 104 and/or a spacing of the teeth 102. As such, the protrusions 140 can include a first protrusion having a first length and a second protrusion having a second length greater than the first length. In some embodiments, the protrusions 140 across a single aligner 120 can have many different lengths (e.g., 2, 3, 5, 10, 20, 30, 40, 50, etc. different lengths). The protrusions 140 across a single aligner 120 can likewise be disposed at many different angles relative to the inner surface 124 of the aligner 120 or to one or more teeth 102 (e.g., 2, 3, 5, 10, 20, 30, 40, 50, etc. different angles). In some embodiments, a subsequent aligner in a treatment plan has more or fewer protrusions than an antecedent aligner, or protrusions having different lengths than the antecedent aligner, or protrusions being disposed at different angles than the antecedent aligner.

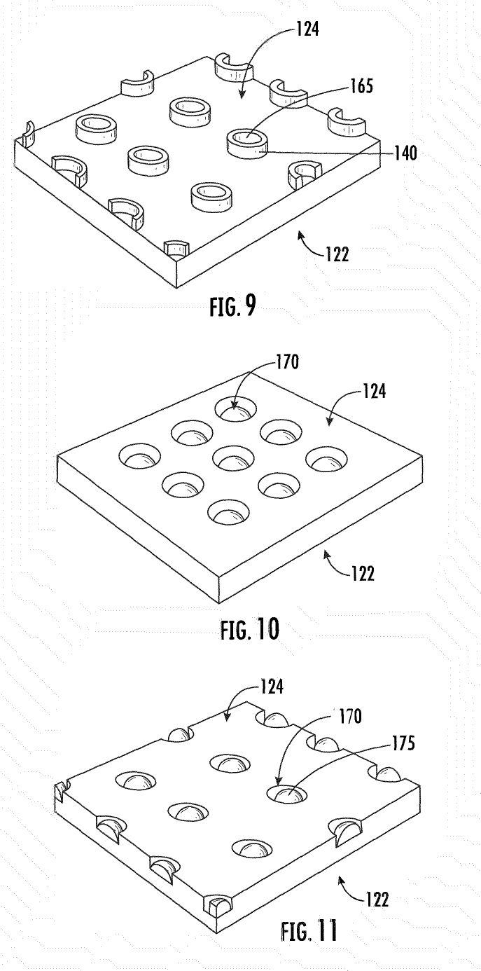

The protrusions 140 may be manufactured according to any desired shape suitable for resisting relative motion between the aligner 120 and the teeth 102. In some embodiments, such as shown in FIGS. 5 and 6, each of the protrusions 140 may be cylindrical in shape, having a substantially circular axial cross-section. In some embodiments, such as shown in FIG. 7, each of the protrusions 140 may have a generally rectangular shape, having a substantially rectangular axial cross-section. In some embodiments, the protrusions 140 may be configured such that the uppermost portion 157 of each individual protrusion 140 is angled or slanted with respect to a longitudinal axis of the protrusion 140, such as shown in FIG. 8. In some embodiments, the protrusions 140 may be structured such that the uppermost portion 157 includes a barb, hook, or other feature configured to engage with the surface 104 of the teeth 102. In some embodiments, a subsequent aligner in a treatment plan has protrusions having a different shape than an antecedent aligner. In some embodiments, the protrusions across a single aligner can have different shapes. In some embodiments, protrusions 140 may be arranged as cavities or recesses within the aligner 120, which may facilitate friction control between the aligner 120 and the teeth.

In various embodiments, each of the protrusions 140 may be oriented in a same direction (e.g., having the same or substantially same first and second angles 150, 155). In some embodiments, the protrusions 140 may be oriented in different directions (e.g., having different first and/or second angles 150, 155). In some embodiments, the regions 126, 128, 130, 132 may be structured such each region has protrusions 140 disposed in a same or similar arrangement. In some embodiments, the regions 126, 128, 130, 132 may each be structured to have different arrangements of the protrusions 140.

In some embodiments, the inner surface 124 (e.g., within the regions 126, 128, 130, 132) may be subjected to one or more surface treatments to increase or otherwise control friction between the inner surface 124 and the surface 104 of the teeth 102. For example, the inner surface 124 may be etched or molded (e.g., micromolded) to form grooves or ridges therein. In some implementations, the grooves or ridges may be disposed in a direction substantially parallel with a primary axis of the teeth 102. In some implementations, the groves or ridges may be oriented in a direction substantially perpendicular to a primary axis of the teeth 102. In some embodiments, the groves or ridges may be arranged according to a predetermined waveform, such as to resemble a sinusoidal wave. In some embodiments, one or more composites and/or particles may be adhered to or molded with the inner surface 124 to increase friction between the inner surface 124 and the surface 104 of the teeth 102. In some embodiments, the aligner 120 may be configured to include both protrusions 140 and one or more grooves, ridges, etching, composites, particles, etc. In some embodiments, the aligner 120 may be used in combination with one or more viscous materials (e.g., gel, paste, etc.) provided to or disposed within the inner contact area 123 that may facilitate an increased resistance to movement of the inner surface 124 relative to the teeth 102. In some embodiments, the one or more viscous materials may be a tooth paste or a whitening gel. For example, the one or more viscous materials may be a whitening gel containing hydrogen peroxide, where the hydrogen peroxide may increase roughness of the surface 104, which may facilitate greater engagement between the inner surface 124 and the teeth 102. In some embodiments, the one or more viscous materials may create a soluble coating on the surface 104 or may alter a morphology or topography of the surface 104 of the teeth 102 to increase grip between the inner surface 124 of the aligner 120 and the teeth 102. In some embodiments, this modification of the roughness of the surface 104 may be temporary, whereas in other embodiments the modification of the surface 104 is permanent. For example, the surface 104 may be configured to react to the one or more viscous materials such that the surface 104 is temporarily modified for a predetermined duration (e.g., 1 hour, 5 hours, 10 hours, 22 hours, 24 hours, 1 week, etc.).

In various embodiments, the aligner 120 or portions thereof may be fabricated or manufactured by the manufacturing system 25 through thermoforming, three-dimensional (3D) printing, casting, molding, micromolding, or any combination thereof. In various embodiments, the inner surface 124 or portions thereof (e.g., the regions 126, 128, 130, and/or 132) may include or consist of materials having high frictional coefficients as compared to the outer surface 105. In various embodiments, the inner surface 124 may include or consist of polydimethylsiloxane (e.g., MED-16), thermoplastic polyurethane elastomer (e.g., Pellethane® 2363-80A), polyurethane resin (e.g., Tecoflex™ EG80A), hard-elastic materials (e.g., Duran®+, Polyethylenterephthalat-Glycol Copolyester), soft-elastic materials (e.g., Erkoflex, ethylvinylacetate), or any other suitable material known in the art. In various embodiments, the inner surface 124 or portions thereof (e.g., the regions 126, 128, 130, and/or 132) may be structured to have a coefficient of friction that is in a range from about 0.7 to about 0.79.

In various embodiments, and as described above, the aligner 120 may be formed such that the geometric variations on the inner surface 124 include one or more recesses or cavities to control (e.g., increase) friction between the inner surface 124 and the surface 104, such as shown in FIGS. 9-11.

As shown in FIG. 9, the inner surface 124 may include a plurality of protrusions 140 extending outwardly from the inner surface 124. Each of the protrusions 140 have a generally cylindrical outer shape and include an internal recess 165 disposed centrally therein. Accordingly, each of the protrusions 140 may have a perforated cylindrical formation. In various embodiments, one or more of the protrusions 140 may additionally or alternatively be substantially rectangular in shape. In various embodiments, one or more of the protrusions 140 may have any other suitable shape on the inner surface 124 (e.g., oblong, pentagonal, hexagonal, etc.). In some embodiments, only a subset of the protrusions 140 may include the internal recess 165. In some embodiments, the protrusions 140 may consist of or include a multitude of shapes and/or sizes. For example, a first subset of the protrusions 140 may be cylindrical having internal recesses 165, a second subset of the protrusions 140 may be rectangular having internal recesses 165, and a third subset of the protrusions may be cylindrical having a height that is less than a height of the first or second subset of protrusions 140.

As shown in FIG. 10, the inner surface 124 may itself include a plurality of cavities or recesses 170 disposed therein. In various embodiments, the recesses 170 may be generally circular, as shown in FIG. 10. In some embodiments, each of the recesses 170 may be structured to have a same (or substantially same) diameter. In some embodiments, each of the recesses 170 may be structured to have a same (or substantially same) depth. In some embodiments, the recesses 170 may have variable diameters and/or depths. For example, a first subset of the recesses 170 may have a first diameter and a first depth, a second subset of the recesses 170 may have a second diameter and second depth, and a third subset of the recesses 170 may have a third diameter and third depth. In various embodiments, the first diameter and first depth may be greater than the second diameter and the second depth. In some embodiments, the first diameter may be greater than the second diameter and the first depth may be less than the second depth. In various embodiments, one or more of the recesses 170 may have a polygonal shape (e.g., rectangular, hexagonal, octagonal, etc.). In some embodiments, a first subset of the recesses 170 may have a generally circular shape and a second subset of the recesses 170 may have a polygonal shape.

As shown in FIG. 11, the recesses 170 may include centrally disposed protrusions 175. The protrusions 175 may be separated from an internal surface of the recesses 170 such that there is a space between an outer surface of each protrusion 175 and an inner surface of the recesses 170. In some embodiments, a base of each protrusion 175 may abut the inner surface of the recess 170 and taper such that an upper portion (e.g., tip) of the protrusion 175 may be spaced from the inner surface of the recess 170. In some embodiments, the protrusions 175 may have a rounded tip. In some embodiments, the protrusions 175 may have a pointed tip. In some embodiments, the protrusions 175 may have a cylindrical shape, rectangular shape, hexagonal shape, conical shape, or any other shape.

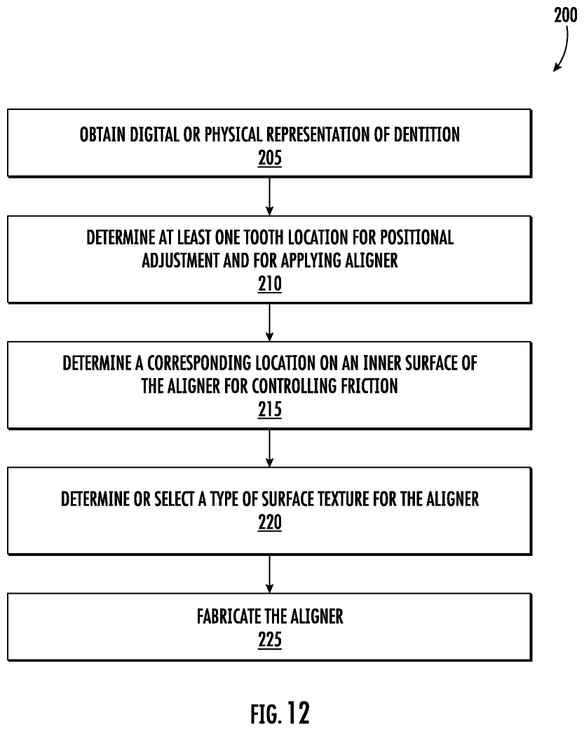

FIG. 12 shows a flow diagram of a method 200 of repositioning the patient's teeth 102. In a first operation 205, the treatment planning computer system 15 may obtain a digital or physical representation of the patient's dentition. For example, the treatment planning computer system 15 may receive a 3D intraoral scan of the patient's mouth, a 3D scan of a dental impression, or a 3D model generated from one or more 2D images received from the patient's smartphone. In a second operation 210, the treatment planning computer system 15 may analyze the digital representation of the teeth 102 to determine at least one tooth among the patient's teeth 102 that is to be repositioned, and determine a location (e.g., region 20, 108, 110 and/or 112) on the teeth 102 for applying a force to cause movement of the teeth 102 or an area of the aligner 120 to grip the teeth 102 and thereby resist movement of the teeth 102 relative to the aligner 120.

In an operation 215, the treatment planning computer system 15 determines corresponding locations (e.g., regions 126, 128, 130, and/or 132) within the aligner 120 to apply one or more forces to the teeth 102 or to control friction between the teeth relative to the aligner 120 (e.g., to resist movement of the teeth 102 relative to the aligner 120) or otherwise modify a surface interaction between the aligner 120 and the teeth 102 to facilitate positional adjustment of the teeth 102. The corresponding locations within the aligner 120 correspond with the locations of the teeth 102 (e.g., region 106, 108, 110 and/or 112) identified for applying the force. In an operation 220, the treatment planning computer system 15 selects a surface modification for the corresponding region to effectuate the necessary force to move the at least one tooth while reducing or eliminating slippage. The surface modification can include a texture or surface type, such as a plurality of protrusions. Selecting the surface modification may include determining a particular configuration of protrusions 140, such as by selecting the height 143, the distance 147, the angles 150, 155, an orientation or configuration of the portion 157, a number of protrusions 140, etc. In some embodiments, selecting the surface modification may additionally or alternatively include selecting a surface treatment for the one or more locations (e.g., region 126, 128, 130 and/or 132), which may include etching, micromolding, 3D printing, composite or particulate deposition, etc.

In an operation 225, the fabrication system 25 (e.g., a manufacturing facility, a molding apparatus, a thermoforming system, a 3D printing device, etc.) may then fabricate the aligner 120 to include the regions 126, 128, 130 and/or 132 having the surface modification as selected by the treatment planning computer system 15.

The embodiments described herein have been described with reference to drawings. The drawings illustrate certain details of specific embodiments that provide the systems, methods and programs described herein. However, describing the embodiments with drawings should not be construed as imposing on the disclosure any limitations that may be present in the drawings.

It should be understood that no claim element herein is to be construed under the provisions of 35 U.S.C. § 112(f), unless the element is expressly recited using the phrase “means for.”

As utilized herein, terms of degree such as “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to any precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that terms such as “exemplary,” “example,” and similar terms, as used herein to describe various embodiments, are intended to indicate that such embodiments are possible examples, representations, or illustrations of possible embodiments, and such terms are not intended to connote that such embodiments are necessarily extraordinary or superlative examples.

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any element on its own or any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the drawings. It should be noted that the orientation of various elements may differ according to other exemplary embodiments, and that such variations are intended to be encompassed by the present disclosure.

As used herein, terms such as “engine” or “circuit” may include hardware and machine-readable media storing instructions thereon for configuring the hardware to execute the functions described herein. The engine or circuit may be embodied as one or more circuitry components including, but not limited to, processing circuitry, network interfaces, peripheral devices, input devices, output devices, sensors, etc. In some embodiments, the engine or circuit may take the form of one or more analog circuits, electronic circuits (e.g., integrated circuits (IC), discrete circuits, system on a chip (SOCs) circuits, etc.), telecommunication circuits, hybrid circuits, and any other type of circuit. In this regard, the engine or circuit may include any type of component for accomplishing or facilitating achievement of the operations described herein. For example, an engine or circuit as described herein may include one or more transistors, logic gates (e.g., NAND, AND, NOR, OR, XOR, NOT, XNOR, etc.), resistors, multiplexers, registers, capacitors, inductors, diodes, wiring, and so on).

An engine or circuit may be embodied as one or more processing circuits comprising one or more processors communicatively coupled to one or more memory or memory devices. In this regard, the one or more processors may execute instructions stored in the memory or may execute instructions otherwise accessible to the one or more processors. The one or more processors may be constructed in a manner sufficient to perform at least the operations described herein. In some embodiments, the one or more processors may be shared by multiple engines or circuits (e.g., engine A and engine B, or circuit A and circuit B, may comprise or otherwise share the same processor which, in some example embodiments, may execute instructions stored, or otherwise accessed, via different areas of memory).

Alternatively or additionally, the one or more processors may be structured to perform or otherwise execute certain operations independent of one or more co-processors. In other example embodiments, two or more processors may be coupled via a bus to enable independent, parallel, pipelined, or multi-threaded instruction execution. Each processor may be provided as one or more suitable processors, application specific integrated circuits (ASICs), field programmable gate arrays (FPGAs), digital signal processors (DSPs), or other suitable electronic data processing components structured to execute instructions provided by memory. The one or more processors may take the form of a single core processor, multi-core processor (e.g., a dual core processor, triple core processor, quad core processor, etc.), microprocessor, etc. In some embodiments, the one or more processors may be external to the apparatus, for example, the one or more processors may be a remote processor (e.g., a cloud based processor). Alternatively or additionally, the one or more processors may be internal and/or local to the apparatus. In this regard, a given engine or circuit or components thereof may be disposed locally (e.g., as part of a local server, a local computing system, etc.) or remotely (e.g., as part of a remote server such as a cloud based server). To that end, engines or circuits as described herein may include components that are distributed across one or more locations.

An example system for providing the overall system or portions of the embodiments described herein might include one or more computers, including a processing unit, a system memory, and a system bus that couples various system components including the system memory to the processing unit. Each memory device may include non-transient volatile storage media, non-volatile storage media, non-transitory storage media (e.g., one or more volatile and/or non-volatile memories), etc. In some embodiments, the non-volatile media may take the form of ROM, flash memory (e.g., flash memory such as NAND, 3D NAND, NOR, 3D NOR, etc.), EEPROM, MRAM, magnetic storage, hard discs, optical discs, etc. In other embodiments, the volatile storage media may take the form of RAM, TRAM, ZRAM, etc. Combinations of the above are also included within the scope of machine-readable media. In this regard, machine-executable instructions comprise, for example, instructions and data which cause a general purpose computer, special purpose computer, or special purpose processing machines to perform a certain function or group of functions. Each respective memory device may be operable to maintain or otherwise store information relating to the operations performed by one or more associated circuits, including processor instructions and related data (e.g., database components, object code components, script components, etc.), in accordance with the example embodiments described herein.

Although the drawings may show and the description may describe a specific order and composition of method steps, the order of such steps may differ from what is depicted and described. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative embodiments. Accordingly, all such modifications are intended to be included within the scope of the present disclosure as defined in the appended claims. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principals of the disclosure and its practical application to enable one skilled in the art to utilize the various embodiments and with various modifications as are suited to the particular use contemplated. Other substitutions, modifications, changes and omissions may be made in the design, operating conditions, and arrangement of the embodiments without departing from the scope of the present disclosure as expressed in the appended claims.

What is claimed is:

1. A device for repositioning a patient's teeth, the device comprising:
a shell having a shape corresponding to the patient's teeth and including an inner surface, wherein the inner surface is configured to engage with the patient's teeth; and
at least one region of the inner surface having a higher frictional coefficient than a remaining portion of the inner surface, wherein the at least one region is configured to apply a force to at least one tooth of the patient's teeth, wherein the at least one region comprises a local variation in geometry relative to a remainder of the inner surface.

2. The device of claim 1, wherein the local variation in geometry comprises a plurality of protrusions.

3. The device of claim 2, wherein the plurality of protrusions extend outwardly from the inner surface, each protrusion of the plurality of protrusions configured to engage with a surface of the at least one tooth.

4. The device of claim 2, wherein each protrusion of the plurality of protrusions is cylindrical in shape, having a substantially circular cross-section.

5. The device of claim 2, wherein each protrusion of the plurality of protrusions is rectangular in shape, having a substantially rectangular cross-section.

6. The device of claim 2, wherein each protrusion of the plurality of protrusions has an uppermost region that is angled with respect to a longitudinal axis of the protrusion.

7. The device of claim 2, wherein each protrusion of the plurality of protrusions is structured to include one or more rows of protrusions.

8. The device of claim 2, wherein each protrusion of the plurality of protrusions is structured to include one or more clusters of protrusions.

9. The device of claim 1, wherein the local variation in geometry comprises a plurality of cavities.

10. The device of claim 1, wherein the local variation in geometry comprises one or more grooves.

11. The device of claim 10, wherein the one or more grooves are oriented in a direction substantially parallel with a primary axis of the at least one tooth.

12. The device of claim 10, wherein the one or more grooves are oriented in a direction substantially perpendicular to a primary axis of the at least one tooth.

13. A system for repositioning a patient's teeth, the system comprising:
an aligner comprising:
an inner surface configured to engage with the patient's teeth and having a shape corresponding to the patient's teeth; and
at least one region of the inner surface having a plurality of protrusions extending outwardly from the inner surface, each protrusion of the plurality of protrusions being configured to apply a force to at least one tooth of the patient's teeth, wherein the plurality of protrusions include a first protrusion having a first length and a second protrusion having a second length greater than the first length.

14. The system of claim 13, wherein the at least one region further includes a plurality of grooves or ridges.

15. The system of claim 13, wherein the plurality of protrusions comprises at least one of a ridge, a stalk, a fiber, or cilia.

16. The system of claim 13, further comprising a viscous material disposed within the aligner, the viscous material configured to increase a coefficient of friction of the inner surface.

17. The system of claim 13, wherein each protrusion of the plurality of protrusions is oriented relative to the inner surface at a first angle on a first side of the protrusion and at a second angle on a second side of the protrusion.

18. A method of repositioning a patient's teeth, the method comprising:
generating, by one or more processors of a treatment planning computer system, a representation of the patient's teeth;
identifying, by the one or more processors based on the representation, a location on at least one tooth of the patient's teeth for applying a force to cause a positional adjustment of the at least one tooth;
determining, by the one or more processors based on the location, a corresponding region on an inner surface of an aligner to be manufactured to apply the force to the location;
selecting, by the one or more processors, a surface modification for the corresponding region, wherein the surface modification comprises a plurality of protrusions; and
generating, by the one or more processors, a treatment plan for the patient to cause the position adjustment of the at least one tooth, wherein the treatment plan includes applying the surface modification to the corresponding region of the aligner.

19. The method of claim 18, further comprising fabricating, by a fabrication system including fabrication equipment, the aligner including the surface modification to the corresponding region of the aligner.

20. The method of claim 18, wherein the plurality of protrusions include a first protrusion having a first length and a second protrusion having a second length greater than the first length.
